(19) United States
(12) Patent Application Publication
(10) Pub. No.: US 2003/0014014 A1
(43) Pub. Date: Jan. 16, 2003

(54) Drug delivery device and method

(76) Inventor: Zvi Nitzan, Zufit (IL)

Correspondence Address: Anthony Castorina, G E Ehrlich 1995, Suite 207, 2001 Jefferson Davis Highway, Arlington, VA, 22202, US

(21) Appl. No.: 10/203,101

(22) PCT Filed: Feb. 8, 2001
(86) PCT No.: PCT/IL01/00129

(51) Int. Cl. A61M 5/178

(52) U.S. Cl. 604/158

(57) ABSTRACT

A drug delivery device for delivering a predetermined drug dose to a body region of an individual, the drug delivery device comprising (a) a reservoir being for containing a liquid preparation of a drug; (b) a drug delivery element being for delivery of the liquid preparation into the body region; and (c) a drug delivery mechanism being capable of fluid communication with one of the reservoir and the drug delivery element at a given time, such that when activated, the drug delivery mechanism cycles through a mode of acquisition of a quantity of the liquid preparation from the reservoir and a subsequent mode of expulsion of the quantity of the liquid preparation acquired from the reservoir, through the drug delivery element into the body region, wherein the predetermined drug dose is delivered by a predetermined number of the cycles.

### FIELD AND BACKGROUND OF THE INVENTION

The present invention relates to a drug delivery device and method, and more particularly, to a skin patch which incorporates a miniaturized drug delivery mechanism capable of cycling drug acquisition and drug discharge steps thus generating pulsatile delivery of an accurate drug dose into a subcutaneous region of the skin even during prolonged periods of use.

The use of drugs for the treatment of various diseases and disorders has been commonly practiced for centuries. Although typically, drugs are delivered at general recommended doses, accurate dosage and extended periods of delivery must sometimes be employed with certain patients or drugs.

In the past, automated drug delivery systems were bulky and oftentimes difficult to operate and as such prolonged drug delivery regimens necessitated patient hospitalization. During the past years, numerous portable drug delivery devices have been designed in an effort to enable accurate dosage and/or prolonged delivery of drugs to those afflicted with disease, chronic illnesses and disabilities. Thus, patients undergoing care and treatment, are no longer rendered immobile but can continue to work, travel and generally enjoy a relatively normal life style while being treated. In instances where prolonged drug administration is desirable, continuous drug flows and infusions at low rates can be beneficially effected by such devices with good results.

Many different types of apparatuses exist in the prior art for administering medication to such patients, including battery powered miniature pumps, hypodermic syringes, implanted reservoirs and dosing devices, skin patches and the like. Some of these devices are capable of automatically or responsively administering a pre-regulated drug dose over a period of time. However, many of these prior art devices are relatively complex and expensive in construction, and/or not always capable of delivering a truly dependable and constant rate of medication over the entire dosage period.

For example, some of the prior art devices are constructed to administer a greater quantity of the drug at the beginning of a dosage cycle than at the end of the cycle. Others are relatively large and cumbersome and are not easily accessible while being used, or are not truly portable, in that the patient is severely restricted in his movements during use of the device.

Many prior art devices of the type described above use the process of pressuring the drug to be administered to the patient, employing for example, elastomeric chambers to hold the drug. Unfortunately, such devices tend to exert a greater pressure on the drug at the beginning of a dispensing cycle as compared to the end, thus resulting in uneven dose administration. Further, contact between the elastomeric chamber wall and the medication can often be a source for contamination in non-sterile environments. Other devices use other means such as pistons, springs or pretensioned elastomeric biasing means for pressurizing the medication. For example, conventionally shaped hypodermic syringes use a spring-propelled piston in a barrel. The barrel is generally cylindrical in configuration, having a straight wall. As the piston moves forward in the barrel, it is subjected to frictional drag. Regulation of the flow of medication in these devices is achieved by the cooperative interaction between the force exerted by the piston on the medication and the flow impeding action produced by a restrictor on the discharge side of the chamber. Consequently, as the spring expands and exerts less force on the piston, the frictional resistance to movement of the piston adversely affects the pressure exerted on the medication being dispensed, resulting in an unwanted changing flow rate as the dispensing cycle progresses. In other words, as the pressure varies and changes during a dispensing cycle, so does the flow rate.

Inaccurate drug delivery is especially true in drug delivery via skin patches. Skin patches, such as transdermal patches, are attached to a skin region of the patient and typically employ mechanisms which enable slow diffusion of a drug onto and through a skin region of the patient. Although some prior art skin patches employ more sophisticated drug delivery mechanisms, such mechanisms are considerably less accurate than some of the prior art delivery mechanisms mentioned above. Since skin patches are typically compact and disposable, delivery mechanisms employed thereby must be inexpensive and miniature.

There is thus a widely recognized need for, and it would be highly advantageous to have, a drug delivery device and method which can deliver a highly accurate dose over prolonged periods of time and which can be employed by compact delivery devices such as skin patches.

### SUMMARY OF THE INVENTION

According to one aspect of the present invention there is provided a drug delivery device for delivering a predetermined drug dose to a body region of an individual, the drug delivery device comprising (a) a reservoir being for containing a liquid preparation of a drug; (b) a drug delivery element being for delivery of the liquid preparation into the body region; and (c) a drug delivery mechanism being capable of fluid communication with one of the reservoir and the drug delivery element at a given time, such that when activated, the drug delivery mechanism cycles through a mode of acquisition of a quantity of the liquid preparation from the reservoir and a subsequent mode of expulsion of the quantity of the liquid preparation acquired from the reservoir, through the drug delivery element into the body region, wherein the predetermined drug dose is delivered by a predetermined number of the cycles.

According to another aspect of the present invention there is provided a skin patch for delivering a predetermined drug dose to a subcutaneous region of an individual, the skin patch comprising (a) a skin attachment element being for attaching the skin patch to a skin region of the individual; (b) a reservoir being connected to or integrally formed with the skin attachment element for containing a liquid preparation of a drug; (c) a needle element being held by the skin attachment element and being dimensioned for delivery of the liquid preparation into the subcutaneous region; and (d) a drug delivery mechanism being capable of fluid communication with one of the reservoir and the needle element at a given time such that when activated, the drug delivery mechanism cycles through a mode of acquisition of a quantity of the liquid preparation from the reservoir and a subsequent mode of expulsion of the quantity of the liquid preparation acquired from the reservoir, through the needle element into the subcutaneous region, wherein the predetermined drug dose is delivered by a predetermined number of the cycles.

According to yet another aspect of the present invention there is provided a method of delivering a predetermined drug close to a body region of an individual, the method comprising the steps of (a) positioning a needle element within the body region; (b) providing a reservoir being for containing a liquid preparation of a drug; (c) providing a drug delivery mechanism being capable of fluid communication with one of the reservoir and the needle element at a given time; and (d) cycling the drug delivery mechanism through a mode of acquisition of a quantity of the liquid preparation from the reservoir and a subsequent mode of expulsion of the quantity of the liquid preparation acquired from the reservoir, through the needle element into the body region, wherein the predetermined drug dose is delivered by a predetermined number of the cycles.

According to further features in preferred embodiments of the invention described below, the skin patch further comprising a power source being for powering the drug delivery mechanism.

According to still further features in the described preferred embodiments the skin patch further comprising a control unit being for controlling the drug delivery mechanism.

According to still further features in the described preferred embodiments the skin patch further comprising a user interface communicating with the control unit.

According to still further features in the described preferred embodiments the skin patch further comprising a data transmitting assembly being in communication with the reservoir, the data transmitting assembly being for acquiring and transmitting to a remote device, data pertaining to the status of the liquid preparation in the reservoir.

According to still further features in the described preferred embodiments the data transmitting assembly also communicates with the drug delivery mechanism and as such, the data transmitted from the data transmitting assembly also includes information pertaining to an operational status of the drug delivery mechanism.

According to still further features in the described preferred embodiments the drug delivery mechanism includes a pump capable of acquiring the quantity of the liquid preparation from the reservoir and expelling the quantity of the liquid preparation acquired from the reservoir, through the needle element.

According to still further features in the described preferred embodiments the pump includes a plunger connected to an actuator, the actuator being for translating the plunger in a first direction for acquiring the quantity of the liquid preparation from the reservoir, and in a second direction for expelling the quantity of the liquid preparation acquired from the reservoir, through the needle element.

According to still further features in the described preferred embodiments the actuator includes a gas generating cell provided with an elastic membrane, such that when the cell generates gas, the membrane expands to translate the plunger in the second direction, and when the gas is released from the cell, the plunger is translated in the first direction.

According to still further features in the described preferred embodiments the gas generating cell includes a zinc-air power cell capable of generating hydrogen gas.

According to still further features in the described preferred embodiments the drug delivery mechanism further includes a switching device being for determining which of the reservoir and the needle element are in fluid communication with the drug delivery mechanism at the given time.

According to still further features in the described preferred embodiments the switching device further serves for determining the mode of the drug delivery mechanism according to the fluid communication thereof at the given time.

According to still further features in the described preferred embodiments the switching device includes a plunger connected to an actuator, the actuator being for translating the plunger in a first direction or in a second and opposing direction.

According to still further features in the described preferred embodiments the drug delivery mechanism includes a first pump being for acquiring the quantity of the liquid preparation from the reservoir, and a second pump being for expelling the quantity of the liquid preparation acquired from the reservoir, through the needle element.

According to still further features in the described preferred embodiments the skin attachment element includes an adhesive.

According to still further features in the described preferred embodiments the needle element is composed of a material selected from the group consisting of stainless steel, carbon fiber, polypropylene and polyurethane.

According to still further features in the described preferred embodiments the needle element includes a plurality of individual micro needles.

According to still further features in the described preferred embodiments the needle element is capable of being positioned in or out of the subcutaneous region when the skin attachment element is attached to the skin region.

The present invention successfully addresses the shortcomings of the presently known configurations by providing a drug delivery device and method which can deliver a highly accurate dose over prolonged periods of time and which can be employed by compact delivery devices such as skin patches.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a cross sectional view of the skin patch according to the present invention;

FIGS. 2A-B are a cross sectional views of one embodiment of the needle element utilized by the skin patch of the present invention, showing the two positions of the needle element;

FIG. 3 is a simplified schematic depiction of a first preferred implementation of the drug delivery mechanism, reservoir and needle element of the skin patch of the present invention;

FIGS. 4A-D depict a full cycle of the drug delivery mechanism of the skin patch of FIG. 3;

FIG. 5 is a simplified schematic depiction of a second preferred implementation of the drug delivery mechanism, reservoir and needle element of the skin patch of the present invention; and

FIGS. 6A-D depict a full cycle of the drug delivery mechanism of the skin patch of FIG. 5.

### DESCRIPTION OF THE PREFERRED EMBODIMENTS

The present invention is of a drug delivery device and method which can be used to deliver an accurate drug dose over a prolonged period of time. Specifically, the present invention is of drug delivery skin patch incorporating a miniaturized drug delivery mechanism which is capable of pulsatile delivery of an accurate drug dose, over an extended time period.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Referring now to the drawings, FIGS. 1-6D illustrate the skin patch for delivering a predetermined drug dose to a subcutaneous region of an individual which is referred to herein as patch 10.

Patch 10 includes a front side surface 12 and a skin contacting backside surface 14, which together form patch body 16. Surfaces 12 and 14 are preferably fabricated from flexible materials which enable surface 12 and/or 14 to conform to the contours of the individuals skin region when patch 10 is applied.

Patch 10 further includes a skin attachment element 18 which serves for attaching patch 10 to a skin region of the individual. Element 18 covers at least a portion of a skin contacting surface 14 of patch 10. To enable attachment to a skin region of the individual, element 12 can include, for example, an adhesive or suction cups which can be used to attach element 12 to the skin, either a single attachment or repeated attachments.

Patch 10 further includes a drug reservoir 20. Reservoir 20 is connected to or integrally formed with skin attachment element 18 and/or surface 14. Reservoir 20 serves for containing a liquid preparation of a drug. As used herein, “a liquid preparation of a drug” includes a solution, suspension or emulsion which includes active concentrations of a drug or a drug which is a liquid per se.

Reservoir 20 is preferably protected by a hard shell 21 which forms a part of front surface 12. Shell 21 protects reservoir 20 from forces which can compress or deform and thus rupture reservoir 20 and/or hamper the operation of patch 10.

Patch 10 further includes a drug delivery or needle element 22 which is held by skin attachment element 18 and/or surface 14. Needle element 22 includes a single needle of between 20-40 gauge in diameter or alternatively a plurality of arrayed micro needles. The single needle or the plurality of micro needles can be fabricated from materials, such as, but not limited to, stainless steel, polyurethane, polypropylene of any composite material which can be made strong and flexible enough to penetrate into the subcutaneous layer of the epidermis without breaking or bending. Needle element 22 can assume a concealed (non-protruding), or a protruding position (shown in FIG. 1). Preferably, prior to application of patch 10, needle element 22 is in the concealed position. Upon application, or at anytime following application of patch 10 onto the skin, needle element 22 is either automatically or manually ejected into the protruding position, in which case needle element 22 preferably extends 2-8 mm from surface 14 and as such is capable of delivering the liquid preparation into the subcutaneous region as is further described hereinunder.

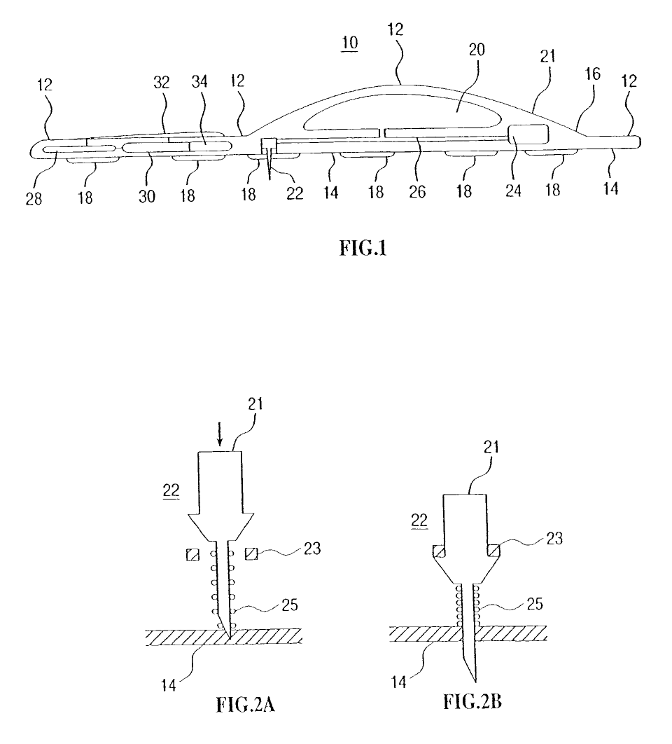

According to one preferred embodiment of the present invention and as specifically shown in FIGS. 2a-b, needle element 22 is ejected from the concealed position (FIG. 2a) to the protruding position (FIG. 2b) by manually depressing a distal portion 21 of needle element 22. This translation locks needle element 22 in the protruding position via a locking mechanism 23 [such as a lock/release ring having a narrower (locking) portion and a wider (releasing) portion] while concomitantly it compresses a return spring 25. Releasing locking mechanism 23 enables return spring 25 to extend thus to retain needle element 22 to its concealed position.

Patch 10 further includes a drug delivery mechanism 24. Mechanism 24 is capable of fluid communication with either reservoir 20 or needle element 22 at a given time; such fluid communication is enabled via a tube or tubes 26. When activated, mechanism 24 cycles through a mode of acquisition of a quantity of the liquid preparation from reservoir 20 and a subsequent mode of expulsion of the quantity of the liquid preparation acquired thereby through needle element 22. As such, mechanism 24 alternates between fluid communication with reservoir 20 at which time mechanism 24 acquires the fluid preparation therefrom, followed by fluid communication with needle element 22 at which time mechanism 24 functions in expelling the liquid preparation acquired thereby through needle element 22 and into, for example, the subcutaneous region of the body.

Thus, each cycle of mechanism 24 functions in delivering a predetermined quantity of the fluid preparation into the subcutaneous region. As a result, mechanism 24 of the present invention can be utilized to deliver any dosage of the drug (as a liquid preparation) by repeating the cycle any desired number of times.

It will be appreciated that this feature of the present invention is particularly advantageous since it enables a very accurate and even delivery of a desired drug dose even throughout an extended period of time. Since the drug dose is delivered by mechanism 24, as a plurality of discrete drug quanta, patch 10 does not suffer from the limitations imposed on prior art drug delivery devices, which limitations include, for example, uneven dose administration especially over prolonged periods of time.

Patch 10 also includes a power source 28 which serves for powering controls of drug delivery mechanism 24.

According to one preferred embodiment of the present invention power source 28 is a battery. Numerous types of miniature batteries which can be incorporated into patch body 12, and utilized to power mechanism 24, are known in the art.

According to a presently preferred embodiment of the present invention battery 28 is a thin flexible battery which engages most of the entire volume of patch body 16. An example of a suitable thin and flexible battery is described in U.S. Pat. No. 5,652,043, which is incorporated herein by reference.

Briefly, the battery described in U.S. Pat. No. 5,652,043 is an open liquid state electrochemical cell which can be used as a primary or rechargeable power supply for various miniaturized and portable electrically powered devices of compact design. The cell comprises a first layer of insoluble negative pole, a second layer of insoluble positive pole and a third layer of aqueous electrolyte, the third layer being disposed between the first and second layers and including (a) a deliquescent material for keeping the open cell wet at all times; (b) an electroactive soluble material for obtaining required ionic conductivity; and, (c) a watersoluble polymer for obtaining a required viscosity for adhering the first and second layers to the first layer. Several preferred embodiments of the battery disclosed in U.S. Pat. No. 5,652,043 include (i) engaging the electrolyte layer in a porous substance, such as, but not limited to, a filter paper, a plastic membrane, a cellulose membrane and a cloth; (ii) having the first layer of insoluble positive pole include manganese-dioxide powder and the second layer of insoluble negative pole include zinc powder; (iii) having the first layer of insoluble negative pole and/or the second layer of insoluble positive pole further include carbon powder; (iv) selecting the electroactive soluble from zinc-chloride, zinc-bromide, zinc-fluoride and potassium-hydroxide; (v) having the first layer of insoluble negative pole include silver-oxide powder and the second layer of insoluble positive pole include zinc powder and the electroactive soluble material is potassium-hydroxide; (vi) having the first layer of insoluble negative pole include cadmium powder and the second layer of insoluble positive pole include nickel-oxide powder and selecting the electroactive soluble material to be potassium-hydroxide; (vii) having the first layer of insoluble negative pole include iron powder and the second layer of insoluble positive pole include nickel-oxide powder and selecting the electroactive soluble material to be potassium-hydroxide; (viii) having the first layer of insoluble negative pole and the second layer of insoluble positive pole include lead-oxide powder, the cell is charged by voltage applied to the poles and the electroactive soluble material is selected in this case to be sulfuric-acid; (ix) the deliquescent material and the electroactive soluble material can be the same material such as zinc-chloride, zinc-bromide, zinc-fluoride and potassium-hydroxide; (x) the deliquescent material is selected from the group consisting of calcium-chloride, calcium-bromide, potassium-biphosphate and potassium-acetate; (xi) the watersoluble polymer can be polyvinylalcohol, poliacrylamide, polyacrylic acid, polyvinylpyrolidone, polyethylenoxide, agar, agarose, starch, hydroxyethylcellulose and combinations and copolymers thereof; (xii) the watersoluble polymer and the deliquescent material can be the same material such as dextrane, dextranesulfate and combinations and copolymers thereof. The cell described in U.S. Pat. No. 5,652,043 preferably includes terminals, each of the terminals being in electrical contact with one of the first and second pole layers. Such terminals can be made, for example, of graphite or a metal, such as iron, nickel, titanium, copper, stainless steel and mixtures thereof. The terminals can be applied to the cell and the entire cell can be manufactured by a suitable printing technology such as, but not limited to, silk print, offset print, jet printing, lamination, materials evaporation or powder dispersion. At least one carbon or graphite based conductive layer can be employed with the cell for improving the electronic conductivity of at least one of the first and second pole layers. Preferred configurations for power source 28 of patch 10 according to the present invention involve those combinations which are devoid of poisonous compounds.

To control the operation of mechanism 24, skin patch 10 further includes a control unit 30 which is in electrical communication with power source 28 and mechanism 24. Control unit 30 can function, for example, in controlling the cycling rate of mechanism 24, and as such the dosage delivered thereby per unit of time. Control unit 30 is preferably also in communication with sensors provided in reservoir 20 and/or needle element 22, such that control unit 30 can monitor the quantity of the liquid preparation stored by patch 10 at any given time.

Patch 10 preferably further includes a user interface 32 which is in communication with control unit 30 and which can be utilized to set the functions of control unit 30. User interface 32 can further, or alternatively, function in displaying the status of mechanism 24 at any given time, or for example, displaying the quantity of the liquid preparation stored thereby at any given time. Thus operation keys and a display preferably form a part of interface 32. In a simpler implementation, one or more LED or the like may be provided to indicate whether the device is currently operating and/or whether a fault has occurred.

Patch 10 further includes a data transmitting assembly 34 which is in communication with control unit 30. Data transmitting assembly 34 serves for acquiring and transmitting to a remote device, data pertaining for example, to the status of the liquid preparation in reservoir 20 or the operation of mechanism 24.

To transmit this data, assembly 34 includes a transmitter, such as, but not limited to, a radio frequency, ultrasound or infrared transmitter. The data transmitted thereby can be digitally encoded or not and can be received by a remote device such as, but not limited to, a personal computer, a cellular telephone and any device capable of receiving and processing transmitted data. It will be appreciated that the received data can be utilized by, for example, a physician to monitor the status of patch 10 when applied to a patient. It will further be appreciated that patch 10 can also include a receiver, such as, but not limited to, a radio frequency, ultrasound or infrared receiver which can receive command signals from a remote device. Such command signals can be used, for example, to switch mechanism 24 on or off, or to change the rate of drug delivery.

Mechanism 24 of the present invention can include one of several devices which are capable of moving a quantum of the liquid preparation out of reservoir 20 and then through needle element 22. However, due to size and expense constraints imposed by patch 10, mechanism 24 must be relatively small and inexpensive to manufacture and yet be able to deliver the liquid preparation in an accurate manner over prolonged periods of time.

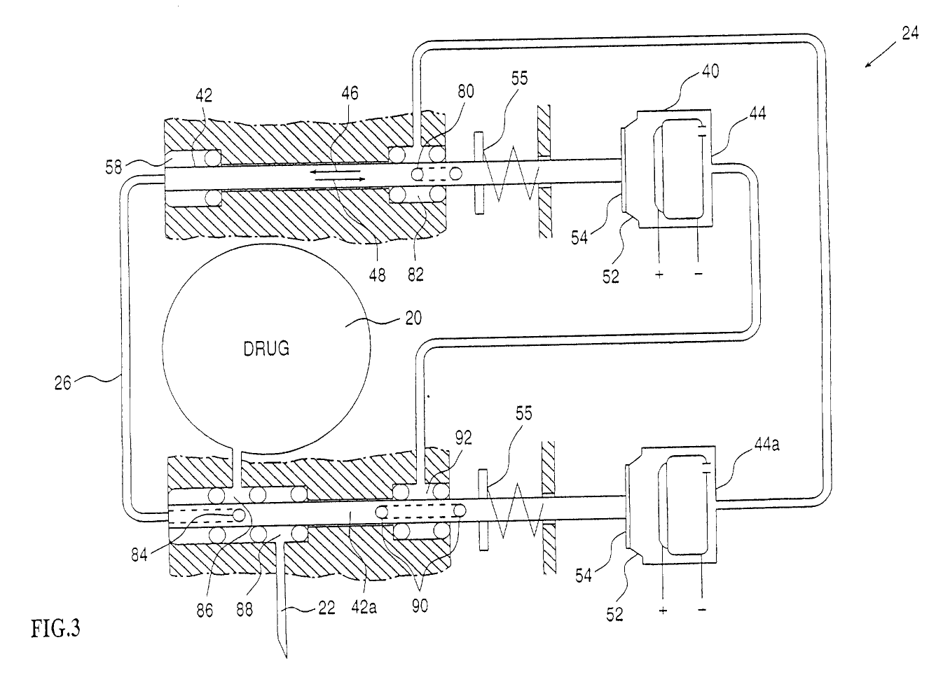

Thus, according to preferred embodiments of the present invention and as specifically shown in FIGS. 3 and 5, mechanism 24 includes a pump 40. Pump 40 includes a plunger 42 which is connected to an actuator 44. Actuator 44 functions in translating plunger 42 in a first direction (as indicated by 46) for acquiring a quantity of the liquid preparation from reservoir 20 into tubing 26 and chamber 58, and in a second direction (as indicated by 48) for expelling a quantity of the liquid preparation acquired from reservoir 20, through needle element 22 and into the subcutaneous region.

Mechanism 24 is preferably actuated by gas pressure differentials, typically originating from a gas-consuming or gas-generating cell such as an appropriate type of battery which is selectively actuated to displace the plunger. In order to generate reciprocating movement from such a cell, two actuating cells 44 and 44a are preferably used, driven out of phase, with each cell operating a system of valves which periodically releases the pressure differential of the other cell. A spring or other resilient element is employed to drive the reverse stroke of each cell when the pressure differential is released.

In order to achieve a particularly compact and reliable system of valves, a preferred type of valve employs a hollow tube or needle with lateral openings which slides within O-ring seals such that the lateral openings are selectively brought into and out of fluid communication with appropriate gas-flow conduits. By selectively sealing certain portions of the hollow tube, each tube may simultaneously provide the functions of multiple valves and/or serve as plunger 42.

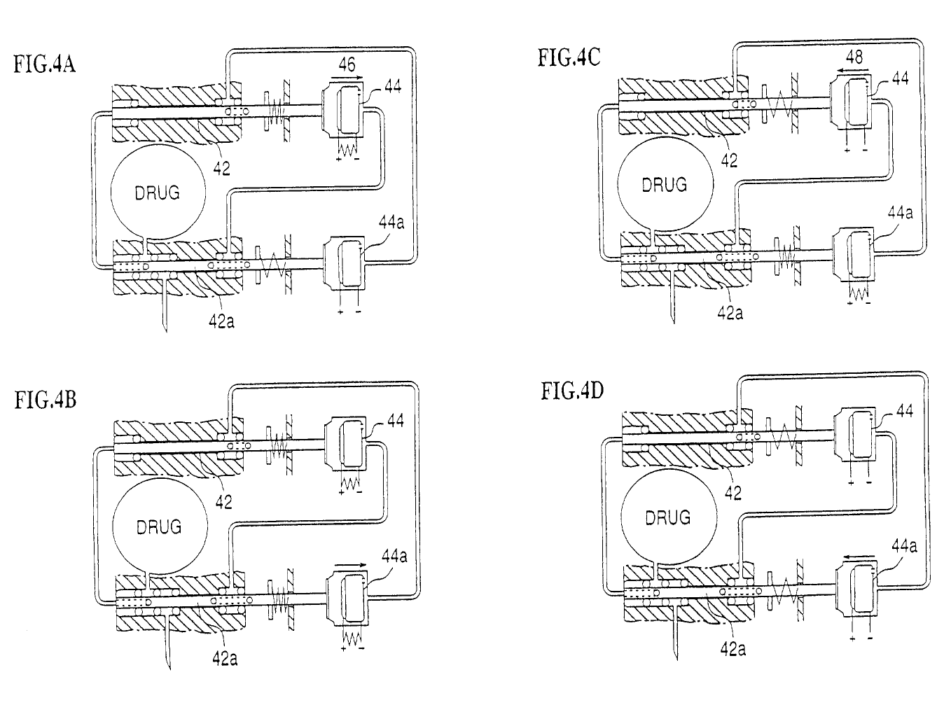

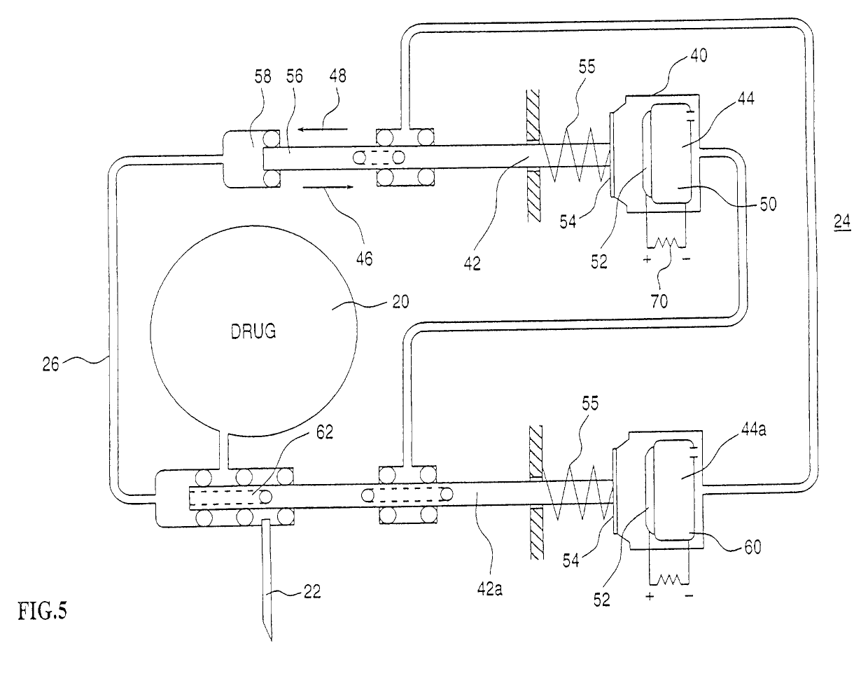

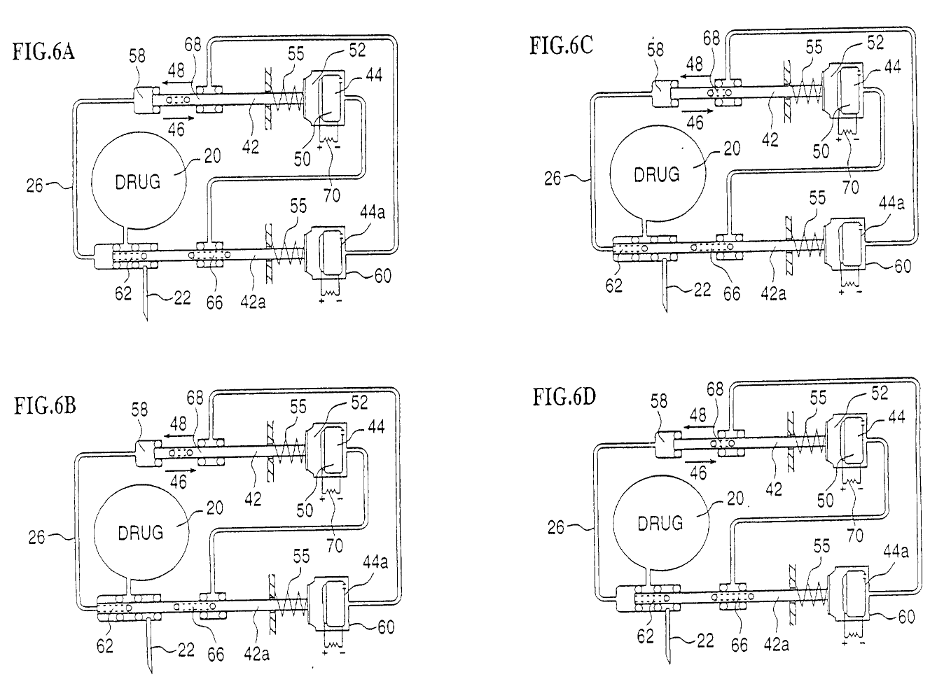

It will be noted that any suitable miniature source of gas pressure or suction may be employed to operate cells 44 and 44a. According to particularly preferred implementations, a pressure differential is generated by a reaction of a battery connected to a load or a driven electrolytic cell located within each actuator cell, thereby rendering the structure extremely simple and compact. The electrochemistry upon which selection of a suitable battery or electrolytic cell which provides net gas generation or consumption is well known and will not be addressed here in detail. By way of particularly preferred but non-limiting examples, a zinc-air battery connected across a load may be used effectively as a gas consuming unit to drive the mechanism by suction, while a similar battery, when starved of oxygen, may be induced to generate hydrogen gas to drive the mechanism by positive pressure. An example of a suction-based implementation will now be described with reference to FIGS. 3-4D, and a gas-generating implementation will be illustrated in FIGS. 5-6D.

Turning now specifically to FIGS. 3-4D, actuators 40 and 44a are here implemented as air-consuming devices deployed within rigid cells 50 which each has an elastic membrane 52 supporting a connecting pad 54 which is mechanically linked to a plunger 42 or 42a. When the air-consuming cell is operated, the gas pressure within the cell drops, thereby sucking membrane 52 inwards so as to draw plunger 42 or 42a against a spring 55. When the pressure differential is released by opening the appropriate valve to allow entrance of more gas, plunger 42 or 42a is returned to the opposite extreme of its motion by action of the spring 55, thereby also extending membrane 52 to its starting position.

The operation and synchronization of the valves will be understood from the cycle sequence shown in FIGS. 4A-4D. FIG. 4A shows the mechanism at the end of its suction motion 46 where the gas pressure in actuator 44 has been reduced sufficiently to withdraw plunger 42 to draw in the drug from reservoir 20 into tube 26 and cavity 58. When plunger 42 reaches the end of its motion, lateral valve openings 80 both lie outside the O-ring release valve assembly 82 for actuator 44a, thereby sealing actuator 44a ready for operation.

Actuator 44a is then brought into operation, thereby displacing plunger 42a as shown in FIG. 4B. This has the effect of moving a first lateral opening 84 within the drug-handling O-ring assembly from the reservoir connection port 86 to the drug delivery port 88. This connects the measured volume of the drug within tube 26 and cavity 58 to delivery needle 22 ready for the delivery stroke. Additionally, towards the end of the movement of plunger 42a, one of lateral valve openings 90 is brought within the O-ring release valve assembly 92 for actuator 44, thereby opening the actuator to the atmosphere via the two lateral openings 90 and the hollow tube therebetween. In order to avoid build-up of nitrogen within the cell, the gas inlet is preferably surrounded by a small supply of oxygen (a few cc's is typically sufficient for prolonged operation). The option of using an oxygen-rich working environment also increases the efficiency of each suction stroke of the cell. Alternatively, the structure may be configured to allow sufficient aeration of the cell during the period that the valve is open to ensure the replenishment of the oxygen content of the air within the cell.

The opening of actuator 44 to the atmosphere allows plunger 42 to perform its delivery stroke 48 under the force of spring 55, thereby forcing a predefined measured quantity of the drug out through needle 22 (FIG. 4C). Additionally, towards the end of the delivery stroke, one of lateral valve openings 80 is brought within the O-ring release valve assembly 82 for actuator 44a, thereby opening the actuator to the atmosphere via the two lateral openings 80 and the hollow tube therebetween. Here too, appropriate precautions are taken to avoid build-up of nitrogen within the cell, as mentioned above.

The opening of actuator 44a to the atmosphere allows plunger 42a to return to its original position under the force of spring 55 as shown in FIG. 4D. This brings lateral opening 84 back into reservoir connection port 86, thereby connecting tube 26 to reservoir 20 ready for withdrawal of the next measured dose of the liquid drug. Lateral valve openings 90 are also removed from release valve assembly 92 for actuator 44, thereby re-sealing actuator 44 ready for repeat operation.

Turning now to FIGS. 5-6D, these show an alternative preferred implementation in which actuators 44 and 44a are implemented as a gas generating cell In this case, when cell 50 generates gas, membrane 52 expands and pushes on connecting pad 54, thus translating plunger 42 in the direction indicated by 48. When the gas is released from cell 50, as is further detailed below, membrane 52 contracts and plunger 42 is reverse translated in the direction indicated by 46, preferably assisted by an expansion of a return spring element 55 which was compressed by the translation of plunger 42 in the direction indicated by 48. This translation is, according to the present invention, reciprocated to thereby repetitively translocate quanta of the liquid preparation from reservoir 20 into chamber 58 of pump 40 and therefrom, through needle element 22, into the subcutaneous region.

According to a preferred embodiment of the present invention gas generating cell 50 includes a zinc-air power cell capable of generating hydrogen gas when connected to a resistor 70.

As mentioned earlier, it will be appreciated that alternative gas generators or sources of gas pressure or suction can also be utilized by the present invention. For example, a heating coil can be used to heat an air volume trapped within cell 50, or electrolysis of water contained within cell 50 can also be utilized by the present invention to generate hydrogen and oxygen gas.

In any case, it will be appreciated that in order to provide translation of plunger 42 in the direction indicated by 46, the gas pressure within cell 44 must be released.

Thus, according to another preferred embodiment of the present invention mechanism 24 further includes a switching device 60 which serves for releasing the gas pressure within cell 50. Device 60 preferably also serves for determining which of reservoir 20 and needle element 22 are in fluid communication with chamber 58 of pump 40 at a given time by determining the position of valve element 62 which is controlled thereby.

According to another preferred embodiment of the present invention device 60 is similar in design to pump 40. Thus, the movement of a plunger 42a of device 60 determines both fluid communication and the mode of operation of pump 40.

According to this embodiment, and as specifically shown in FIGS. 6A-6D which describe, in sequence, a single cycle of mechanism 24, pump 40 and device 60 of mechanism 24 function in a complementary manner closely analogous to that of FIGS. 4A-4D described above. As specifically shown in FIG. 6A, when gas is generated in cell 50, membrane 52 expands and plunger 42 translates in the direction indicated by 48 to thereby push a quantity of the liquid preparation from chamber 58 and tubing 26 through needle element 22 into the subcutaneous region. As specifically shown in FIG. 6B, following full translation of plunger 42 of pump 40, device 60 which functions in a similar manner to pump 40, is operated. Gas generated in a cell 44a of device 60 translates a plunger 42a thereof and as a result valve 62 communicates between reservoir 20 and chamber 58 and the gas is released from cell 44 of pump 40 via a valve 66. This enables membrane 52 of pump 40 to retract and assisted by return spring 55 to translate plunger 42 of pump 40 in the direction indicated by arrow 46. As a result, a quantum of liquid preparation is translocated from reservoir 20 into chamber 58 by a suction action applied by plunger 42 of pump 40. This suction activates valve 68 which releases the gas trapped within cell 44a of device 60 as is specifically shown in FIGS. 6C and 6D.

Once chamber 58 fills with the liquid drug preparation the cycle completes and now a quantum of the liquid preparation can be delivered to the subcutaneous region by reactivating pump 40 as shown in FIG. 6A. It will be appreciated that the movement of plunger 42a of device 60 in the direction indicated by 46 as shown in FIGS. 6C and 6D also re communicates chamber 58 with needle element 22 via valve 62, such that when step 6A is effected again, the fluid preparation will be delivered from needle element 22 into the subcutaneous region.

Thus, the present invention provides a drug delivery skin patch which includes a drug delivery mechanism capable of delivering an accurate drug dose throughout a prolonged period of time. In addition, since the drug delivery mechanism of the present invention is exceptionally inexpensive to fabricate and can be miniaturized beyond the size required for the skin patch, without loss of function or accuracy, such a mechanism can also be incorporated into a variety of drug delivery devices such as, for example, miniature intrabody positioned drug delivery devices.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications cited herein are incorporated by reference in their entirety. Citation or identification of any reference in this application shall not be construed as an admission that such reference is available as prior art to the present invention.

What is claimed is:

1. A drug delivery device for delivering a predetermined drug dose to a body region of an individual, the drug delivery device comprising:
(a) a reservoir being for containing a liquid preparation of a drug;
(b) a drug delivery element being for delivery of said liquid preparation into the body region;
(c) a drug delivery mechanism being capable of fluid communication with one of said reservoir and said drug delivery element at a given time, such that when activated, said drug delivery mechanism cycles through a mode of acquisition of a quantity of said liquid preparation from said reservoir and a subsequent mode of expulsion of said quantity of said liquid preparation acquired from said reservoir, through said drug delivery element into the body region, wherein the predetermined drug dose is delivered by a predetermined number of said cycles.

2. A skin patch for delivering a predetermined drug dose to a subcutaneous region of an individual, the skin patch comprising:
(a) a skin attachment element being for attaching the skin patch to a skin region of the individual;
(b) a reservoir being connected to or integrally formed with said skin attachment element for containing a liquid preparation of a drug;
(c) a needle element being held by said skin attachment element and being dimensioned for delivery of said liquid preparation into the subcutaneous region;
(d) a drug delivery mechanism being capable of fluid communication with one of said reservoir and said needle element at a given time, such that when activated, said drug delivery mechanism cycles through a mode of acquisition of a quantity of said liquid preparation from said reservoir and a subsequent mode of expulsion of said quantity of said liquid preparation acquired from said reservoir, through said needle element into the subcutaneous region, wherein the predetermined drug dose is delivered by a predetermined number of said cycles.

3. The skin patch of claim 2, further comprising a power source being for powering said drug delivery mechanism.

4. The skin patch of claim 2, further comprising a control unit being for controlling said drug delivery mechanism.

5. The skin patch of claim 4, further comprising a user interface communicating with said control unit.

6. The skin patch of claim 2, further comprising a data transmitting assembly being in communication with said reservoir, said data transmitting assembly being for acquiring and transmitting to a remote device, data pertaining to the status of said liquid preparation in said reservoir.

7. The skin patch of claim 6, wherein said data transmitting assembly also communicates with said drug delivery mechanism and as such, said data transmitted from said data transmitting assembly also includes information pertaining to an operational status of said drug delivery mechanism.

8. The skin patch of claim 2, wherein said drug delivery mechanism includes a pump capable of acquiring said quantity of said liquid preparation from said reservoir and expelling said quantity of said liquid preparation acquired from said reservoir, through said needle element.

9. The skin patch of claim 8, wherein said pump includes a plunger connected to an actuator, said actuator being for translating said plunger in a first direction for acquiring said quantity of said liquid preparation from said reservoir, and in a second direction for expelling said quantity of said liquid preparation acquired from said reservoir, through said needle element.

10. The skin patch of claim 9, wherein said actuator includes a gas generating cell provided with an elastic membrane, such that when said cell generates gas, said membrane expands to translate said plunger in one of said first and said second directions, and when said gas is released from said cell, said plunger is translated in another of said first and said second directions.

11. The skin patch of claim 10, wherein said gas generating cell includes a zinc-air power cell capable of generating hydrogen gas.

12. The skin patch of claim 9, wherein said actuator includes a gas consuming cell provided with an elastic membrane, such that when said cell consumes gas, said membrane is displaced to translate said plunger in one of said first and said second directions, and when said gas is released from said cell, said plunger is translated in another of said first and said second directions.

13. The skin patch of claim 12, wherein said gas consuming cell includes a zinc-air power cell capable of reacting with oxygen gas.

14. The skin patch of claim 2, wherein said drug delivery mechanism further includes a switching device being for determining which of said reservoir and said needle element are in fluid communication with said drug delivery mechanism at said given time.

15. The skin patch of claim 14, wherein said switching device further serves for determining said mode of said drug delivery mechanism according to said fluid communication thereof at said given time.

16. The skin patch of claim 15, wherein said switching device includes a plunger connected to an actuator, said actuator being for translating said plunger in a first direction or in a second and opposing direction.

17. The skin patch of claim 2, wherein said skin attachment element includes an adhesive.

18. The skin patch of claim 2, wherein said needle element is composed of a material selected from the group consisting of stainless steel, carbon fiber, polypropylene and polyurethane.

19. The skin patch of claim 2, wherein said needle element includes a plurality of individual micro needles.

20. The skin patch of claim 2, wherein said needle element is capable of being positioned in or out of said subcutaneous region when said skin attachment element is attached to said skin region.

21. A method of delivering a predetermined drug dose to a body region of an individual, the method comprising the steps of:
(a) positioning a needle element within the body region;
(b) providing a reservoir being for containing a liquid preparation of a drug;
(c) providing a drug delivery mechanism being capable of fluid communication with one of said reservoir and said needle element at a given time; and
(d) cycling said drug delivery mechanism through a mode of acquisition of a quantity of said liquid preparation from said reservoir and a subsequent mode of expulsion of said quantity of said liquid preparation acquired from said reservoir, through said needle element into the body region, wherein the predetermined drug dose is delivered by a predetermined number of said cycles.

22. The method of claim 21, wherein said needle element, said reservoir and said drug delivery mechanism are integrated within a drug delivery skin patch.
